SEQUENCE LISTING (31 sequences)

SEQ ID NO: 1 - DNA, 417 bp, Homo sapiens
cgtcttttccagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcat

SEQ ID NO: 2 - DNA, 17 bp, Artificial
  Description of artificial sequence Synthetic primer
cacgctgcagacgatcc

SEQ ID NO: 3 - DNA, 21 bp, Artificial
  Description of artificial sequence Synthetic primer
ccccaatccatcttctactct

SEQ ID NO: 4 - DNA, 17 bp, Artificial
  Description of artificial sequence Synthetic primer
tcccacatccggctatg

SEQ ID NO: 5 - DNA, 22 bp, Artificial
  Description of artificial sequence Synthetic primer
gatggggaagtagtgatcgtag

SEQ ID NO: 6 - DNA, 313 bp, Homo sapiens
aggcaagaagcacggaggcctgtggtggacctacctgttcagcctcatcttcaagctcatcattgagttcctcttcctctacctgctgcacactctctggcatggcttcaatatgccgcgcctggtgcagtgtgccaacgtggccccctgccccaacatcgtggactgctacattgcccgacctaccgagaagaaaatcttcacctacttcatggtgggcgcctccgccgtctgcatcgtactcaccatctgtgagctctgctacctcatctgccacagggtcctgcgaggcctgcacaaggacaagcctcga

SEQ ID NO: 7 - DNA, 19 bp, Artificial
  Description of artificial sequence Synthetic primer
cgtggactgctacattgcc

SEQ ID NO: 8 - DNA, 15 bp, Artificial
  Description of artificial sequence Synthetic primer
atgttggggcagggg

SEQ ID NO: 9 - DNA, 240 bp, Homo sapiens
tcatgatagtttagaaaagatacatctgtagaaaggttgaatatttaccgtttctaaaatggaaccttgaccctcttgagatttcacttggttctgtagatagagtatagcatcatggaccgtcaaaaagaatgtgtcctttctaatgttgtcgtcaaagaacccgcattgctccagcttttctatcacataatcttcaaaagaaagacacaatgttttgttagttcctaggaaaagaaa

SEQ ID NO: 10 - DNA, 15 bp, Artificial
  Description of artificial sequence Synthetic primer
cgtcatttcgggagt

SEQ ID NO: 11 - DNA, 15 bp, Artificial
  Description of artificial sequence Synthetic primer
ctaagcagccattcc

SEQ ID NO: 12 - DNA, 300 bp, Homo sapiens
aatgtataattcagaaaaccagaaccttaccacccgcagtgatctcactccaacaacgtccaggaaagatatagctccacagtcaagcacaaggctatggattggcactttgggaacgttcactttgactggaagctcagagttccaatccacttgaatctctatttccttggttgggatatcaagttcctccagatcttcaatatcctcatcaggctcaaaagcattatttgttgaaacagcatcacttatgatgccattctaaacgaagaaaacactgtcaacttaattgtcaaagat

SEQ ID NO: 13 - DNA, 15 bp, Artificial
  Description of artificial sequence Synthetic primer
ccctgactctgctgg

SEQ ID NO: 14 - DNA, 15 bp, Artificial
  Description of artificial sequence Synthetic primer
cactggcaatcagga

SEQ ID NO: 15 - DNA, 336 bp, Homo sapiens
acgctggttgagatttttcaaaatattggtgataccaatcttgctgatttcactgctggattgctcaccattgtcgtctgtatggcagttaaggaattaaatgatcggtttagacacaaaatcccagtccctattcctatagaagtaattgtggtaagtagaatatgtagttagaaagttcagcattatttggttgacaaacaaggaattattaaaaccaatggagtttttaacatcttttgttttatttcagacgataattgctactgccatttcatatggagccaacctggaaaaaaattacaatgctggcattgttaaatccatcccaagggg

SEQ ID NO: 16 - DNA, 20 bp, Artificial
  Description of artificial sequence Synthetic primer
tggcagtagcaattatcgtc

SEQ ID NO: 17 - DNA, 20 bp, Artificial
  Description of artificial sequence Synthetic primer
tttcatatggagccaacctg

SEQ ID NO: 18 - DNA, 114 bp, Homo sapiens
gaattcattgcctttgggatcagcaacatcttctcaggattcttctcttgttttgtggccaccactgctctttcccgcacggccgtccaggagagcactggaggaaagacacag

SEQ ID NO: 19 - DNA, 17 bp, Artificial
  Description of artificial sequence Synthetic primer
ccactgctctttcccgc

SEQ ID NO: 20 - DNA, 24 bp, Artificial
  Description of artificial sequence Synthetic primer
caagagaagaatcctgagaagatg

SEQ ID NO: 21 - DNA, 231 bp, Homo sapiens
aatggcatcataagtgatgctgtttcaacaaataatgcttttgagcctgatgaggatattgaagatctggaggaacttgatatcccaaccaaggaaatagagattcaagtggattggaactctgagcttccagtcaaagtgaacgttcccaaagtgccaatccatagccttgtgcttgactgtggagctatatctttcctggacgttgttggagtgagatcactgcgggtg

SEQ ID NO: 22 - DNA, 19 bp, Artificial
  Description of artificial sequence Synthetic primer
ttcctggacgttgttggag

SEQ ID NO: 23 - DNA, 23 bp, Artificial
  Description of artificial sequence Synthetic primer
gatatagctccacagtcaagcac

SEQ ID NO: 24 - DNA, 218 bp, Homo sapiens
aaaacattgtgtctttcttttgaagattatgtgatagaaaagctggagcaatgcgggttctttgacgacaacattagaaaggacacattctttttgacggtccatgatgctatactctatctacagaaccaagtgaaatctcaagagggtcaaggttccattttagaaacggtaaatattcaacctttctacagatgtatcttttctaaactatcatg

SEQ ID NO: 25 - DNA, 20 bp, Artificial
  Description of artificial sequence Synthetic primer
tcttgagatttcacttggtt

SEQ ID NO: 26 - DNA, 20 bp, Artificial
  Description of artificial sequence Synthetic primer
gttccattttagaaacggta

SEQ ID NO: 27 - DNA, 16 bp, Artificial
  Description of artificial sequence Synthetic linker
gtctcatccctgcgtg

SEQ ID NO: 28 - DNA, 16 bp, Artificial
  Description of artificial sequence Synthetic linker
cacgcagggtacgtgt

SEQ ID NO: 29 - DNA, 16 bp, Artificial
  Description of artificial sequence Synthetic primer
gtctcatccctgcgtg

SEQ ID NO: 30 - DNA, 16 bp, Artificial
  Description of artificial sequence Synthetic primer
acacgtaccctgcgtg

SEQ ID NO: 31 - DNA, 32 bp, Artificial
  Description of artificial sequence Synthetic auxiliary sequence
gtgcgtccctactctgtgtgcatgggacgcac

(19) United States
(12) Patent Application Publication
(10) Pub. No.: US 2015/0307942 A1
(43) Pub. Date: Oct. 29, 2015

(54) METHOD AND A KIT FOR NON-INVASIVELY DETECTING FETAL DEAFNESS PATHOGENIC GENE MUTATIONS

(71) Applicant: BERRY GENOMICS CO., LTD., Beijing (CN)

(72) Inventors: Yiqian Liu, Beijing (CN); Xianchao Guo, Beijing (CN); Yong Fu, Beijing (CN); Yugang Hu, Beijing (CN); Tiancheng Li, Beijing (CN); Jianguang Zhang, Beijing (CN)

(73) Assignee: BERRY GENOMICS CO., LTD., Beijing (CN)

(21) Appl. No.: 14/693,578

(22) Filed: Apr. 22, 2015

(30) Foreign Application Priority Data
Apr. 23, 2014 (CN) 201410174277.6

(51) Int. Cl. C12Q 1/68

(52) U.S. Cl. CPC C12Q 1/6883; C12Q 2600/16; C12Q 2600/156; C12Q 2600/158

(57) ABSTRACT

The present invention is directed to a method, kit and primers for detecting fetal deafness pathogenic gene mutations. The method of the invention comprises: (a) designing primers according to the pre-determined mutation loci of deafness pathogenic genes; (b) extracting plasma DNAs in a pregnant woman; (c) connecting the extracted plasma DNAs with pre-amplification linkers to obtain connected products; (d) PCR pre-amplifying the connected product to obtain pre-amplified products; (e) cyclizing the pre-amplified products to obtain cyclised DNAs; (f) PCR amplifying the cyclised DNAs using the designed primers to obtain amplified products; and (g) high throughput sequencing the amplified products and analyzing the mutations of the fetal deafness pathogenic genes. The invention can effectively determine whether the pre-determined loci on deafness pathogenic genes have been mutated as well as the mutation type.

### REFERENCE TO RELATED APPLICATIONS

This application claims the benefit of priority to Chinese Patent Application No. CN 201410174277.6, filed on Apr. 23, 2014, the entire contents of which are incorporated herein by reference.

### FIELD OF INVENTION

The present invention relates to genetic diagnosis field. More specifically, the present invention is directed to a method for detecting fetal deafness pathogenic gene mutations. The present invention also relates to a kit and uses thereof for detecting fetal deafness pathogenic gene mutations.

### BACKGROUND OF INVENTION

Deafness is a common disease resulting in disability and affecting health in humans, and is also one of the most common genetic diseases in clinic. According to statistics, there is one with dysaudia among every 1000 new-born babies over the world (Steel K P., “New interventions in hearing impairment,” BMJ, 2000, 320(4):622-625). Many reasons can result in deafness, while the genetic factor is the main one. Hearing disability occurred at the time of born or before 3 years old is called pre-lingual hearing impairment, at least half of which is caused by genetic deficiency. In a large number of patients with delayed hearing loss, many patients developed said disease due to their genetic deficiency, or due to increased susceptibility to the environment caused by genetic deficiency and polymorphism. It is estimated that there are totally more than 100 non-syndrome genetic deafness genes globally. In view of the domestic research progress, most of the pathogenicity loci found in China are on GJB2, GJB3, SLC26A4 and mitochondria 12SrRNA.

Brief introductions of GJB2, GJB3, and SLC26A4 are provided herein for better understanding. GJB2 gene: this gene is located on autosomal 13q11-12 region, and the DNA full length is 4804 bp, including 2 exons. The coding region is 678 bp, and encodes a connexion Connexin26 consisting of 266 amino acid residues, which belongs to beta-2 protein that is part of the potassium circulation pathway. GJB2 gene mutation is the most common cause for genetic deafness, and the deafness resulted from GJB2 gene mutation is pre-lingual, bilateral, and symmetric deafness, which varies a lot in terms of the degree of hearing loss. It may range from mild to extremely severe, while most is severe or extremely severe deafness. In Chinese population, the main types of GJB2 gene mutation are 235delC, 299-300delAT, 176-191del16 and the like, accounting for over 80% of the populations with GJB2 gene mutation. GJB3 gene: this gene is located on autosomal 1q33-35 region, has 2 exons, and encodes a connexion Connexin 31 comprising 270 amino acids. GJB3 gene mutation can lead to autosomal genetic non-syndrome deafness, dominant or recessive, and is considered to be associated with high frequency hearing loss. SLC26A1 gene is located on autosomal 7q31 region, has 21 exons, and encodes a multipass transmembrane protein Pendrin consisting of 780 amino acid residues, which belongs to the transporter family that mainly relates with iodine/chloride ion transportation and plays an important role in maintaining the balance of the body ion compositions. Recently, many studies abroad demonstrate that SLC26A4 is close related with Pendred syndrome (enlarged vestibular aqueduct or accompanied with nerve deafness of inner ear malformation and goiter) and large vestibular aqueduct syndrome (LVAS). Among the numerous mutations, many are presented in both Pendred syndrome and LVAS. Thus, mutations at the same loci may result in different clinic performances. There are many types of SLC26A4 gene mutations, while the mutation frequencies of 281C>T, 589G>A, IVS7-2A>G, 1174A>T(N392Y), 1226G>A, 1229C>T(T410M), 1975G>C, 2027T>A(L676Q), 2168A>G(HIS723ARG) and IVS15+5G>A are as high as 82.51%.

With the development of science and technology, many deafness patients of new-born babies are diagnosed using methods of Sanger sequencing, gene chip and protein detection. There is also prenatal detection based on invasive diagnosis of fetal deafness pathogenic genes. However, non-invasively detection of fetal deafness pathogenic genes has not been achieved yet.

### SUMMARY OF INVENTION

The inventor explored a method for detecting fetal deafness pathogenic gene mutations (genotype) using fragment DNA from venous blood of a pregnant woman based on the second generation high-throughput sequencing technology. After the discovery of embryo DNA in maternal blood, it is possible to non-invasively diagnose and detecting fetal chromosomal abnormalities and gene mutations directly (Lo Y M et al., (1997) “Presence of fetal DNA in maternal plasma and serum,” Lancet, 350:485-487). The Illumina products are the best among the second generation high-throughput sequencing technologies, in which two representative products are Miseq and Hiseq, one is known for sequencing length, and the other is known for sequencing throughput. Miseq sequencer is used in the present invention. However, the amount of embryo DNA in blood is low, and how to determine the related fetal genotypes quickly and accurately is still a technical problem to be solved.

The technical problem to be solved in the present invention is how to non-invasively detecting the genotypes of fetal deafness pathogenic genes using the venous blood of a pregnant woman. Accordingly, the first object of the present invention is to provide a method that is capable of effectively detecting gene mutations of pre-determined mutation loci on deafness pathogenic genes in a fetus through the use of plasma DNA samples, comprising the following steps:
(a) designing primers according to the pre-determined mutation loci of deafness pathogenic genes;
(b) extracting plasma DNAs in a pregnant woman;
(c) connecting the extracted plasma DNAs with pre-amplification linkers to obtain connected products;
(d) PCR pre-amplifying the connected product to obtain pre-amplified products;
(e) cyclizing the pre-amplified products to obtain cyclised DNAs;
(f) PCR amplifying the cyclised DNAs using the designed primers to obtain amplified products; and
(g) high throughput sequencing the amplified products and analyzing the mutations of the fetal deafness pathogenic genes.

According to one preferred embodiment of the invention, the pre-determined locus is at least one gene mutation selected from the 22 loci of GJB2 gene, GJB3 gene and SLC26A4 gene.

According to one preferred embodiment of the invention, the primers are a pair of primers that are backward extended.

According to one preferred embodiment of the invention, the backward extended pair of primers is designed to aim at exon 2 of GJB2, exon 2 of GJB3, or exon 3, exon 5, exon 7, intron 7, exon 8, exon 10, exon 17 or exon 19 of SLC26A4.

According to one preferred embodiment of the invention, the backward extended pair of primers consist of backward extended pair of primers that are adjacent to a group of disease detection loci (e.g. high frequency mutation loci).

According to one preferred embodiment of the invention, the backward extended pair of primers contains universal primer region suitable for different high-throughput sequencing platforms.

According to another preferred embodiment of the invention, the sequences of the backward extended pair of primers are as follows respectively:

| | |
|---|---|
| GJB2-F1 (SEQ ID NO: 2): | CACGCTGCAGACGATCC |
| GJB2-R1 (SEQ ID NO: 3): | CCCCAATCCATCTTCTACTCT |
| GJB2-F2 (SEQ ID NO: 4): | TCCCACATCCGGCTATG |
| GJB2-R2 (SEQ ID NO: 5): | GATGGGGAAGTAGTGATCGTAG |
| GJB3-F (SEQ ID NO: 7): | CGTGGACTGCTACATTGCC |
| GJB3-R (SEQ ID NO: 8): | ATGTTGGGGCAGGGG |
| PDS3-F (SEQ ID NO: 10): | CGTCATTTCGGGAGT |
| PDS3-R (SEQ ID NO: 11): | CTAAGCAGCCATTCC |
| PDS5-F (SEQ ID NO: 13): | CCCTGACTCTGCTGG |
| PDS5-R (SEQ ID NO: 14): | CACTGGCAATCAGGA |
| PDS7-F2 (SEQ ID NO: 16): | TGGCAGTAGCAATTATCGTC |
| PDS7-R2 (SEQ ID NO: 17): | TTTCATATGGAGCCAACCTG |
| PDS10-F (SEQ ID NO: 19): | CCACTGCTCTTTCCCGC |
| PDS10-R (SEQ ID NO: 20): | CAAGAGAAGAATCCTGAGAAGATG |
| PDS17-F (SEQ ID NO: 22): | TTCCTGGACGTTGTTGGAG |
| PDS17-R (SEQ ID NO: 23): | GATATAGCTCCACAGTCAAGCAC |
| PDS19-F (SEQ ID NO: 25): | TCTTGAGATTTCACTTGGTT |
| PDS19-R (SEQ ID NO: 26): | GTTCCATTTTAGAAACGGTA. |

According to one preferred embodiment of the invention, one pair of the backward extended pair of primers detects one or more loci.

According to one preferred embodiment of the invention, detection of the 22 loci of GJB2 gene, GJB3 gene and SLC26A4 gene is accomplished in one PCR using 9 pairs of primers.

According to one preferred embodiment of the invention, the linkers are barcode (multiple sequence labelled) linkers.

According to one preferred embodiment of the invention, there are at least two base differences between the linkers.

According to one preferred embodiment of the invention, the linkers are partially matched Y-type linkers.

According to one preferred embodiment of the invention, the method used in the cyclization of step c) is a splint cyclization, wherein single stand DNAs complementary to both ends of the pre-amplified DNA are used as splints; and close of the ring is completed by a heat-resistant Taq ligase.

According to one preferred embodiment of the invention, the cyclization of step c) is multiple reactions of a single system circulation consisting of DNA denaturation, splint DNA annealing and connecting.

According to one preferred embodiment of the invention, the method of the invention further comprises digesting the uncyclized linear DNA.

According to one preferred embodiment of the invention, the method of the invention detects the genotype of fetal deafness pathogenic genes using fragment DNA from venous blood of a pregnant woman.

According to one preferred embodiment of the invention, the deafness pathogenic genes have insertion, deletion, substitution or gene fusion mutations.

The second object of the present invention is to provide a kit for non-invasively detecting fetal deafness pathogenic gene mutations, comprising:

reagents for extracting plasma DNAs, a DNA cyclase, primers and reagents for amplifying target DNAs.

According to one preferred embodiment of the invention, the kit further comprises primers and reagents for pre-amplifying pre-determined loci of deafness pathogenic genes.

According to one preferred embodiment of the invention, the kit further comprises reagents for high throughput sequencing.

According to one preferred embodiment of the invention, the plasma DNAs are connected with linkers, wherein the linkers are barcode (multiple sequence labelled) linkers.

The third object of the present invention is to provide a use of primers designed against pre-determined loci of deafness pathogenic genes in the preparation of diagnosing reagents or kits for non-invasively detecting fetal deafness pathogenic gene mutations, characterized in that the diagnosing reagents or kits are applicable to a method for non-invasively detecting fetal deafness pathogenic gene mutations comprising the following steps:
(a) designing primers according to the pre-determined mutation loci of deafness pathogenic genes;
(b) extracting plasma DNAs in a pregnant woman;
(c) connecting the extracted plasma DNAs with pre-amplification linkers to obtain connected products;
(d) PCR pre-amplifying the connected product to obtain pre-amplified products;
(e) cyclizing the pre-amplified products to obtain cyclised DNAs;
(f) PCR amplifying the cyclised DNAs using the designed primers to obtain amplified products; and
(g) high throughput sequencing the amplified products and analyzing the mutations of the fetal deafness pathogenic genes.

### BRIEF DESCRIPTION OF FIGURES

FIG. 1 shows the design of primers used in the present invention.

FIG. 2 shows the structure of connected products of the present invention.

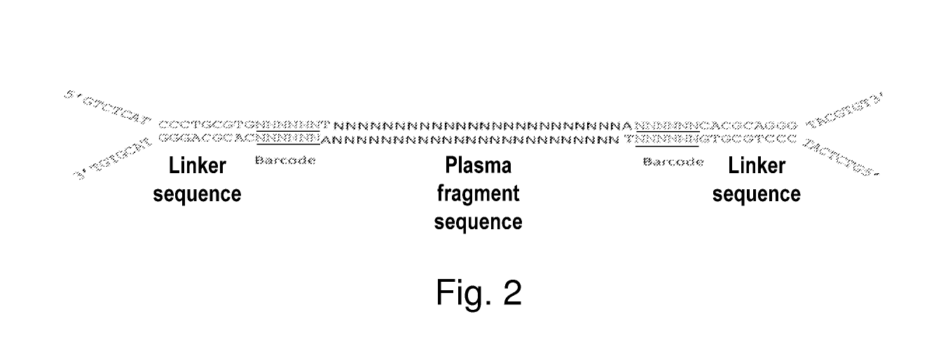

FIG. 3 shows auxiliary sequence and linker sequence of the present invention, wherein the arrow points to the position needed to be linked by Taq DNA ligase.

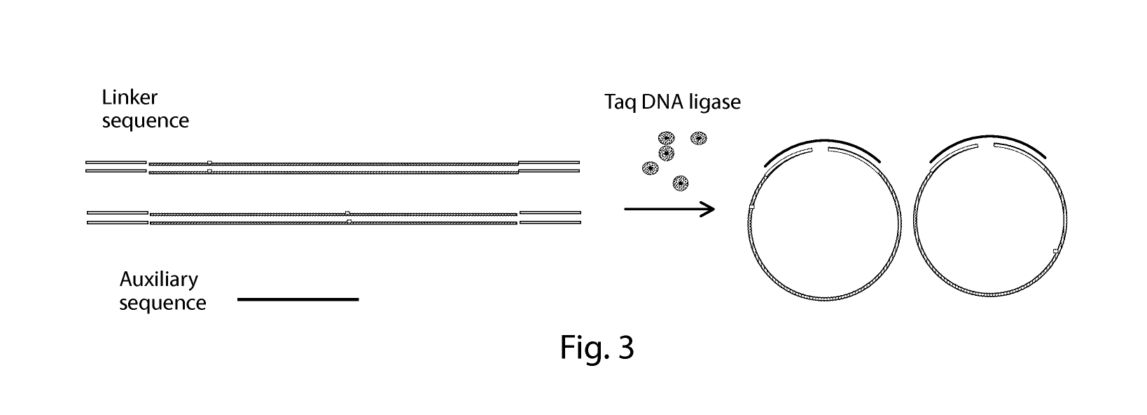

FIG. 4 shows one preferred embodiment of the method of the invention, illustrating the scheme of constructing a library.

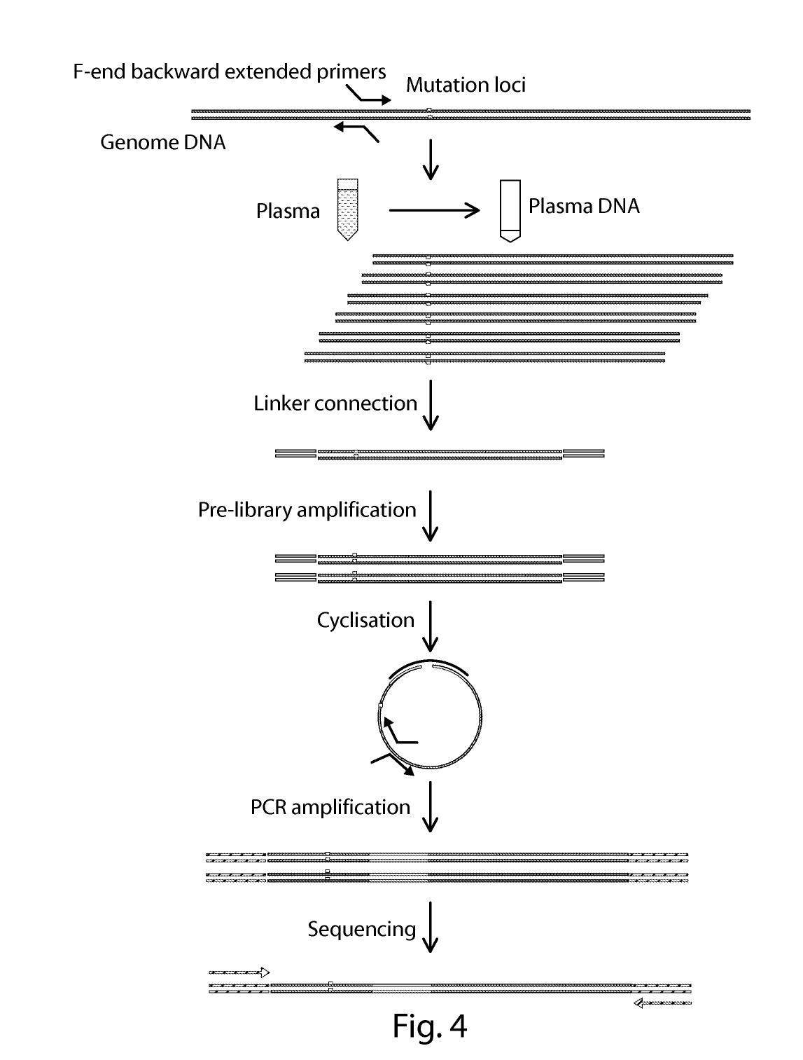

### DETAILED DESCRIPTION OF THE INVENTION

The first object of the present invention is to provide a method for detecting gene mutations of pre-determined mutation loci of deafness pathogenic genes in a fetus through the use of plasma DNA samples, comprising the following steps:
(a) designing primers according to the pre-determined mutation loci of deafness pathogenic genes;
(b) extracting plasma DNAs in a pregnant woman;
(c) connecting the extracted plasma DNAs with pre-amplification linkers to obtain connected products;
(d) PCR pre-amplifying the connected product to obtain pre-amplified products (pre-library);
(e) cyclizing the pre-amplified products to obtain cyclised DNAs;
(f) PCR amplifying the cyclised DNAs using the designed primers to obtain amplified products; and
(g) high throughput sequencing the amplified products and analyzing the mutations of the fetal deafness pathogenic genes.

Traditional methods for detecting fragment DNA are mainly performed by PCR amplifying the regions to be tested before detection. As the PCR primers are on both ends of the regions to be tested, the regions to be tested are required to be complete. However, the regions to be tested in most fragment DNAs are incomplete, as the fragment DNAs are produced by random cleavage. Accordingly, the number of fragment DNAs that can be used as amplification templates is few, and is difficult to be detected. In the invention, the adaptation range of the primer amplification and the effective amount of templates are largely increased by cyclizing the fragment DNAs, thereby improving the detection sensitivity of the fragment DNA dramatically. A more important aspect of the invention is to label the initial template using the start and terminal positions of the fragment DNA as well as the 6 different bases on 3′ end of the linker while improving the utility of the template. Thus, whether the pre-determined loci in mother and fetus have been mutated as well as the type of mutations can be effectively determined. Another important aspect of the invention is to detect multiple loci to be tested simultaneously, e.g., to detect the 22 high frequency pathogenic loci in a sample to be tested at one time using the Multi-PCR technology.

The detection method of the invention can be used to detect fragment DNA comprising mutations, such as homozygous mutation and heterozygous mutation; or base deletion, insertion or substitution. “Fragment DNA” of the invention refers to short fragment DNA with the length of about 166 bp that is formed by random cleavage of the genome DNA of an organism.

In sum, template cyclization of the invention makes the available DNA templates more than that of conventional PCR, thereby improving detection sensitivity. Labelling initial templates using the start and terminal positions of the fragment DNA as well as the 6 different bases of Barcode on 3′ end of the linker makes it possible to effectively reduce the sequence to a template sequence through sequencing before PCR amplification, thus decreasing the sequence bias resulted from PCR amplification, and determining whether the pre-determined loci in mother and fetus have been mutated as well as the type of mutations more accurately. Meanwhile, 9 pairs of backward extended primers are designed and optimized against high frequency mutation loci of the deafness pathogenic genes GJB2, GJB3, and SLC26A4, and the 22 high frequency pathogenic loci in a sample to be tested are detected at one time using the Multi-PCR technology.

### Experiment Design of a Deafness Example

### 1. Primer Design of the Pre-Determined Mutation Loci on Deafness Pathogenic Genes.

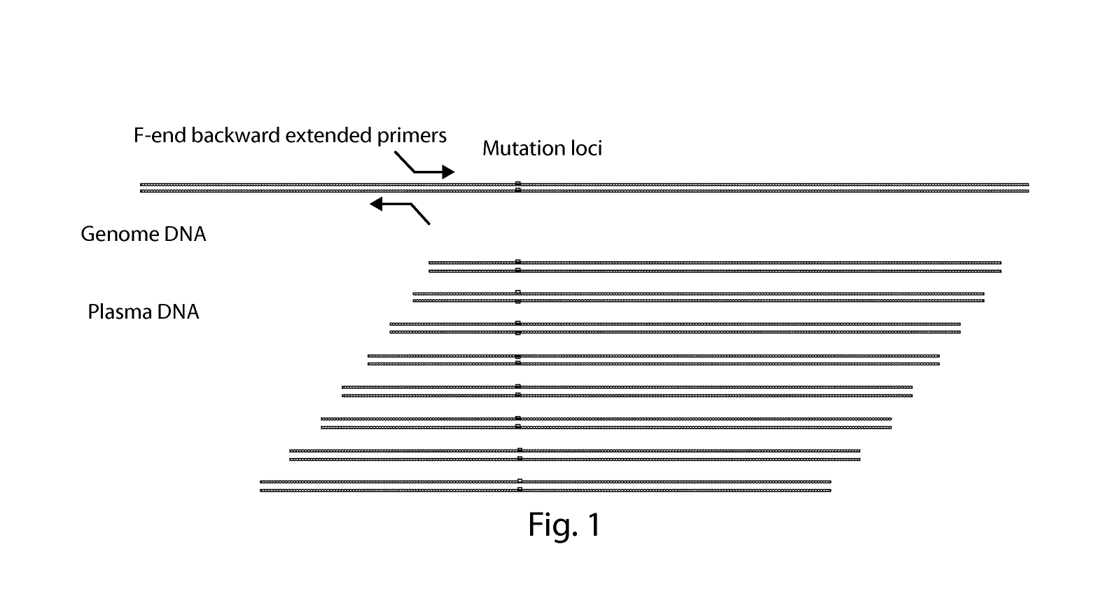

To achieve the detection of mutation genotypes of the pre-determined loci of fetal deafness pathogenic genes in maternal venous blood, the mutation of the pre-determined loci of the invention is at least one gene mutation selected from the 22 loci of GJB2 gene, GJB3 gene, and SLC26A4 gene, preferably, the mutation of GJB2 gene is at least one mutation selected from 35delG, 109G>A(VAL37ILE)167delT, 176-191del16, 235delC and 299_{—}300delAT; the mutation of GJB3 gene is at least one mutation selected from 421A>G(ILE141VAL), 421-423delATT, 497A-G(ASN166SER), 538C>T(ARG180TER), 547G>A(GLU183LYS) and 580G>A(ALA194THR); and the mutation of SCL26A4 gene is at least one mutation selected from 281C>T, 589G>A, IVS7-2A>G, 1174A>T(N392Y), 1226G>A, 1229C>T(T410M), 1975G>C, 2027T>A(L676Q), 2162C>T(T721M), 2168A>G(HIS723ARG) and IVS15+5G>A. The tested loci do not include mitochondria C1494T and A1555G mutation loci, as the amount of mitochondria DNA in plasma is very low. More preferably, the pairs of primers are designed to extend backward for detection of pre-determined mutation loci, and the F-end primer is closer to the tested loci, as shown in FIG. 1. More preferably, the 22 loci on GJB2, GJB3, and SLC26A4 genes are detected in one PCR using 9 pairs of primers by the mean of Multi-PCR that solves the multi-point detection. The specific sequences tested in GJB2, GJB3, and SLC26A4 genes and the primers are as follows:

Backward extended amplification primers in the target region aimed at exon 2 of GJB2.

The sequence of GJB2 exon 2 is as follows
(SEQ ID NO: 1):
CGTCTTTTCCAGAGCAAACCGCCCAGAGTAGAAGATGGATTGGGGCACGC
TGCAGACGATCCTGGGGGGTGTGAACAAACACTCCACCAGCATTGGAAAG
ATCTGGCTCACCGTCCTCTTCATTTTTCGCATTATGATCCTCGTTGTGGC
TGCAAAGGAGGTGTGGGGAGATGAGCAGGCCGACTTTGTCTGCAACACCC
TGCAGCCAGGCTGCAAGAACGTGTGCTACGATCACTACTTCCCCATCTCC
CACATCCGGCTATGGGCCCTGCAGCTGATCTTCGTGTCCACGCCAGCGC
TCCTAGTGGCCATGCACGTGGCCTACCGGAGACATGAGAAGAAGAGGA
AGTTCATCAAGGGGGAGATAAAGAGTGAATTTAAGGACATCGAGGAGAT
CAAAACCCAGAAGGTCCGCAT
GJB2-F1 (SEQ ID NO: 2):
CACGCTGCAGACGATCC
GJB2-R1 (SEQ ID NO: 3):
CCCCAATCCATCTTCTACTCT
GJB2-F2 (SEQ ID NO: 4):
TCCCACATCCGGCTATG
GJB2-R2 (SEQ ID NO: 5):
GATGGGGAAGTAGTGATCGTAG

Backward extended amplification primers in the target region aimed at exon 2 of GJB3.

The sequence of GJB3 exon 2 is as follows
(SEQ ID NO: 6):
AGGCAAGAAGCACGGAGGCCTGTGGTGGACCTACCTGTTCAGCCTCATCT
TCAAGCTCATCATTGAGTTCCTCTTCCTCTACCTGCTGCACACTCTCTGG
CATGGCTTCAATATGCCGCGCCTGGTGCAGTGTGCCAACGTGGCCCCCTG
CCCCAACATCGTGGACTGCTACATTGCCCGACCTACCGAGAAGAAAATC
TTCACCTACTTCATGGTGGGCGCCTCCGCCGTCTGCATCGTACTCACCAT
CTGTGAGCTCTGCTACCTCATCTGCCACAGGGTCCTGCGAGGCCTGCAC
AAGGACAAGCCTCGA
GJB3-F (SEQ ID NO: 7):
CGTGGACTGCTACATTGCC
GJB3-R (SEQ ID NO: 8):
ATGTTGGGGCAGGGG

Backward extended amplification primers in the target region aimed at exon 3 of SLC26A4.

The sequence of SLC26A4 exon 3 is as follows
(SEQ ID NO: 9):
TCATGATAGTTTAGAAAAGATACATCTGTAGAAAGGTTGAATATTTACCG
TTTCTAAAATGGAACCTTGACCCTCTTGAGATTTCACTTGGTTCTGTAGA
TAGAGTATAGCATCATGGACCGTCAAAAAGAATGTGTCCTTTCTAATGTT
GTCGTCAAAGAACCCGCATTGCTCCAGCTTTTCTATCACATAATCTTCA
AAAGAAAGACACAATGTTTTGTTAGTTCCTAGGAAAAGAAA
PDS3-F (SEQ ID NO: 10):
CGTCATTTCGGGAGT
PDS3-R (SEQ ID NO: 11):
CTAAGCAGCCATTCC

Backward extended amplification primers in the target region aimed at exon 5 of SLC26A4.

The sequence of SLC26A4 exon 5 is as follows
(SEQ ID NO: 12):
AATGTATAATTCAGAAAACCAGAACCTTACCACCCGCAGTGATCTCACTC
CAACAACGTCCAGGAAAGATATAGCTCCACAGTCAAGCACAAGGCTATGG
ATTGGCACTTTGGGAACGTTCACTTTGACTGGAAGCTCAGAGTTCCAATC
CACTTGAATCTCTATTTCCTTGGTTGGGATATCAAGTTCCTCCAGATCTT
CAATATCCTCATCAGGCTCAAAAGCATTATTTGTTGAAACAGCATCACTT
ATGATGCCATTCTAAACGAAGAAAACACTGTCAACTTAATTGTCAAAGAT
PDS5-F (SEQ ID NO: 13):
CCCTGACTCTGCTGG
PDS5-R (SEQ ID NO: 14):
CACTGGCAATCAGGA

Backward extended amplification primers in the target region aimed at exon 7, intron 7 and exon 8 of SLC26A4.

The sequence of SLC26A4 exon 7, intron 7 and
exon 8 are as follows (SEQ ID NO: 15):
ACGCTGGTTGAGATTTTTCAAAATATTGGTGATACCAATCTTGCTGATTT
CACTGCTGGATTGCTCACCATTGTCGTCTGTATGGCAGTTAAGGAATT
AAATGATCGGTTTAGACACAAAATCCCAGTCCCTATTCCTATAGAAGT
AATTGTGGTAAGTAGAATATGTAGTTAGAAAGTTCAGCATTATTTGGTT
GACAAACAAGGAATTATTAAAACCAATGGAGTTTTTAACATCTTTTGTT
TTATTTCAGACGATAATTGCTACTGCCATTTCATATGGAGCCAACCTG
GAAAAAAATTACAATGCTGGCATTGTTAAATCCATCCCAAGGGG
PDS7-F2 (SEQ ID NO: 16):
TGGCAGTAGCAATTATCGTC
PDS7-R2 (SEQ ID NO: 17):
TTTCATATGGAGCCAACCTG

Backward extended amplification primers in the target region aimed at exon 10 of SLC26A4.

The sequence of SLC26A4 exon 10 is as follows
(SEQ ID NO: 18):
GAATTCATTGCCTTTGGGATCAGCAACATCTTCTCAGGATTCTTCTCTTG
TTTTGTGGCCACCACTGCTCTTTCCCGCACGGCCGTCCAGGAGAGCA
CTGGAGGAAAGACACAG
PDS10-F (SEQ ID NO: 19):
CCACTGCTCTTTCCCGC
PDS10-R (SEQ ID NO: 20):
CAAGAGAAGAATCCTGAGAAGATG

Backward extended amplification primers in the target region aimed at exon 17 of SLC26A4.

The sequence of SLC26A4 exon 17 is as follows
(SEQ ID NO: 21):
AATGGCATCATAAGTGATGCTGTTTCAACAAATAATGCTTTTGAGCCTGA
TGAGGATATTGAAGATCTGGAGGAACTTGATATCCCAACCAAGGAAAT
AGAGATTCAAGTGGATTGGAACTCTGAGCTTCCAGTCAAAGTGAACGT
TCCCAAAGTGCCAATCCATAGCCTTGTGCTTGACTGTGGAGCTATATC
TTTCCTGGACGTTGTTGGAGTGAGATCACTGCGGGTG
PDS17-F (SEQ ID NO: 22):
TTCCTGGACGTTGTTGGAG
PDS17-R (SEQ ID NO: 23):
GATATAGCTCCACAGTCAAGCAC

Backward extended amplification primers in the target region aimed at exon 19 of SLC26A4.

The sequence of SLC26A4 exon 19 is as follows
(SEQ ID NO: 24):
AAAACATTGTGTCTTTCTTTTGAAGATTATGTGATAGAAAAGCTGGAGCA
ATGCGGGTTCTTTGACGACAACATTAGAAAGGACACATTCTTTTTGACG
GTCCATGATGCTATACTCTATCTACAGAACCAAGTGAAATCTCAAGAG
GGTCAAGGTTCCATTTTAGAAACGGTAAATATTCAACCTTTCTACAGA
TGTATCTTTTCTAAACTATCATG
PDS19-F (SEQ ID NO: 25):
TCTTGAGATTTCACTTGGTT
PDS19-R (SEQ ID NO: 26):
GTTCCATTTTAGAAACGGTA

### 2. Linker Design of the Plasma DNA

To achieve quantified template detection of the loci of the deafness pathogenic genes, the invention employed barcode (multiple sequence labelled) linkers to label the sequences of plasma DNA. The labelling is obtained using the start and terminal positions of the fragment DNA as well as the 6 different bases on 3′ end of the linker. The above labelling achieves two objectives: one is to quantitatively label the initial templates of the tested loci; the other is to exclude contamination between different samples in the same batch. To achieve the first objective, the number of combination types of a group of barcode linkers has to be much bigger than the maximal number of the templates with the same start and same terminal positions. For instance, in 1 ml plasma, the maximal number of the templates with the same start and same terminal positions is approximately 10, and the number of combination types of each group of barcode linkers of the invention is 10, thus the number of random combinations of upstream and downstream of the linkers is 100. The combination number of 100 is much bigger than the template number of 10, thus each template is ensured to link to different type of barcode linkers. To achieve the second objectives, various combinations of barcode linkers are designed in the invention. There are totally 16 groups of barcode linkers without repetition between each group of linkers, thus a maximal of 16 samples can be detected in one experiment. More preferably, there are at least two base differences between different barcode linkers, thus to reduce the possibility of mistake. Correction function of the barcode linkers achieves quantification and eliminates contamination. Thus, whether the pre-determined loci in mother and fetus have been mutated as well as the type of mutations can be effectively determined. The specific linker and primer sequences are as follows:

Linker design, need to be annealed to a double strand:

ssCycAB-1(SEQ ID NO: 27):
GTCTCATCCCTGCGTG(NNNNNNT)
ssCycAB-2(SEQ ID NO: 28):
p(NNNNNN)CACGCAGGGTACGTGT

wherein N can be any amino acid.
pre-library amplification primers:

| | |
|---|---|
| ssCycUniprimer-F(SEQ ID NO: 29): | GTCTCATCCCTGCGTG |
| ssCycUniprimer-R(SEQ ID NO: 30): | ACACGTACCCTGCGTG |

### 3. Cyclisation of the Pre-Library

Traditional methods for detecting fragment DNA are mainly performed by PCR amplifying the regions to be tested before detection. As the PCR primers are designed on both ends of the regions to be tested, the regions to be tested are required to be complete. However, the regions to be tested in most fragment DNAs are incomplete, as the fragment DNAs are produced by random cleavage. Accordingly, the number of fragment DNAs that can be used as amplification templates is few, and is difficult to be detected by PCR. The invention uses plasma DNA fragment (about 166 bp) to achieve quantified template detection of the loci on the deafness pathogenic genes. First, the fragment DNA is cyclized. As long as the cleavage point is not in the template of the backward extended primers, the amplification can be conducted. Thus, the adaptation range of the primer amplification and the effective amount of templates are increased, and the detection sensitivity of the fragment DNA is also improved dramatically. After connection of the plasma DNA and linkers, the pre-library is amplified and is subsequently cyclized. Cyclization of the pre-library can be auto-connection to a ring. The invention selects single strand splint cyclization with specific auxiliary sequences. Preferably, the linkers connected to plasma fragment DNA are partially matched Y-type linkers, wherein the length of matched base that next to barcode is 9 bp, and the length of unmatched base is 7 bp. Preferably, a single strand DNA that is complementary to the Y-type linkers is used in assisting the cyclization. The linkers and auxiliary sequences are complementary to each other to form double strands, wherein the length of the auxiliary sequence is 32 bp. Preferably, the ends between the linker sequences are connected by Taq DNA Ligase (MO208L) from NEB.

### Auxiliary Sequence:

Bridge (SEQ ID NO: 31):
GTGCGTCCCTACTCTGTGTGCATGGGACGCAC

### Specific Embodiments

### Experiment Protocols

### 1. Extraction of Plasma DNA

Plasma DNA was extracted from 1-2 ml plasma by QIAamp Circulating Nucleic Acid Kit (CAT No. 55114). DNA was eluted in 45 μl elution buffer, wherein 2 μl is used for concentration detection by Qubit.

### 2. End-Filing and Addition of A on the Plasma DNA

The reaction mixture was prepared as shown in Table 1.

TABLE 1

| | |
|---|---|
| T4 DNA polymerase buffer (10 X) | 5 μl |
| Plasma DNA | 40.5 μl |
| Taq polymerase | 0.5 μl |
| T4 DNA polymerase | 2.0 μl |
| 10 mM dNTP | 2.0 μl |
| Total volume | 50 μl |

### Reaction on a PCR Machine:

### 37° C.: 20 min

### 72° C.: 20 min

4° C.: maintain

The product with A addition was purified on a column, dissolved in 25 μl Buffer EB, and eluted twice.

3. Connection with the Linkers

The reaction mixture was prepared as shown in Table 2.

TABLE 2

| | |
|---|---|
| DNA | 22 μl |
| 2X Quick Ligase Buffer | 25 μl |
| 7.5 μM CycAB linker | 2 μl |
| T4 DNA ligase (HC) | 1 μl |
| Total volume | 50 μl |

### Reaction on a PCR Machine:

### 20° C.: 15 min

### 65° C.: 10 min

4° C.: maintain

### 4. Construction of Pre-Library

4.1 PCR (100 μl system), the reaction mixture was prepared as shown in Table 3.

TABLE 3

| | |
|---|---|
| Phusion PCR Master Mix (2X) | 50 μl |
| CycUniprimer (F-10 μM, R-35 μM) | 2 μl |
| Products connected with linkers | 50 μl |
| Total volume | 100 μl |

### 4.2 PCR Programs are Shown in Table 4.

TABLE 4

| | | |
|---|---|---|
| 98° C. | 30 s | 1 cycle |
| 98° C. | 10 s | 14 cycles |
| 65° C. | 30 s | |
| 72° C. | 30 s | |
| 72° C. | 5 min | 1 cycle |
| 4° C. | maintain | |

### 5. Phosphorylation and Cyclization

### 5.1 The Phosphorylation and Cyclization System was Prepared as Shown in Table 5.

TABLE 5

| | |
|---|---|
| ATP | 0.4 μl |
| T4 PNK | 0.5 μl |
| 10X Taq Ligase Buffer | 4 μl |
| Bridge (10 μM) | 4 μl |
| Pre-library products | 12 μl |
| Taq Ligase | 2 μl |
| EB | 17.1 μl |
| Total volume | 50 μl |

### 5.2 5.2 PCR Programs are Shown in Table 6.

TABLE 6

| | | |
|---|---|---|
| 37° C. | 30 min | |
| 95° C. | 30 s | 30 cycles |
| 50° C. | 2 min | |
| 4° C. | maintain | |

### 6. Exonuclease Digestion

6.1 Components as Shown in Table 7 were Added in the Reaction System of Step 5 in Order.

TABLE 7

| | |
|---|---|
| Exo I | 1 μl |
| Exo III | 1 μl |

Reaction on a PCR machine at 37° C. for 10 min

| | |
|---|---|
| PK (3 mg/ml) | 1 μl |

### 6.2 Reaction Conditions are as Shown in Table 8.

TABLE 8

| | |
|---|---|
| 50° C. | 10 min |
| 99° C. | 4 min |
| 4° C. | maintain |

### 7. PCR Screening of the Target Region Using the Backward Extended Primers

The detection of the 22 loci of GJB2 gene, GJB3 gene and SLC26A4 gene was performed in one PCR using 9 pairs of primers by means of Multi-PCR, which solve multi-point detection.

### 7.1 PCR Reaction System was Prepared as Shown in Table 9

TABLE 9

| | |
|---|---|
| Phusion PCR Master Mix (2x) | 50 μl |
| Primer Mix (0.5-2 μM for each) | 4 μl |
| Cyclized DNA | 43 μl |
| EB | 3 μl |
| Total volume | 50 μl |

### 7.2 PCR Reaction Conditions are as Shown in Table 10

TABLE 10

| | | |
|---|---|---|
| 98° C. | 30 s | 1 cycle |
| 98° C. | 10 s | 25 cycles |
| 60° C. | 30 s | |
| 72° C. | 30 s | |
| 72° C. | 5 min | 1 cycle |
| 4° C. | maintain | |

After PCR reaction, the product was immediately purified using 90 μl XP Beads (0.9×), and then dissolved in 26 μl Buffer EB.

### 8. Generation of the Final Library

### 8.1 PCR Reaction System was Prepared as Shown in Table 11.

TABLE 11

| | |
|---|---|
| Phusion PCR Master Mix (2x) | 25 μl |
| Illumina-Nextera-F (25 μM) | 0.5 μl |
| Index Primer (25 μM) | 0.5 μl |
| Screened PCR products | 24 μl |
| Total volume | 50 μl |

8.2 PCR Reaction Conditions were as Shown in Table 12

TABLE 12

| | | |
|---|---|---|
| 98° C. | 30 s | 1 cycle |
| 98° C. | 10 s | 25 cycles |
| 65° C. | 30 s | |
| 72° C. | 30 s | |
| 72° C. | 5 min | 1 cycle |
| 4° C. | maintain | |

After PCR reaction, 10 μl product was analysed by electrophoresis on 2% agarose gel, while other products were purified and recycled using 0.8× XP Beads, and were finally dissolved in 22 μl EB Buffer, which were used in a subsequent Q-PCR as the final library. The size of the final library is 320 bp (used in calculating QPCR concentration), and it should mix with Read1 Sequencing Primer from NEXTERA for sequencing. 300 bp double-end sequencing was performed using Miseq from Illumina.

The sample was plasma from a pregnant woman. Preferably, the mutation of the pre-determined loci is at least one gene mutation selected from the 22 loci of GJB2 gene, GJB3 gene, and SLC26A4 gene. More preferably, the mutation of GJB2 gene is at least one mutation selected from 35delG, 109G>A(VAL37ILE)167delT, 176-191del16, 235delC and 299_{—}300 delAT; the mutation of GJB3 gene is at least one mutation selected from 421A>G(ILE141VAL), 421-423delATT, 497A-G(ASN166SER), 538C>T(ARG180TER), 547G>A(GLU183LYS) and 580G>A(ALA194THR); and the mutation of SCL26A4 gene is at least one mutation selected from 281C>T, 589G>A, IVS7-2A>G, 1174A>T(N392Y), 1226G>A, 1229C>T(T410M), 1975G>C, 2027T>A(L676Q), 2162C>T(T721M), 2168A>G(HIS723ARG) and IVS15+5G>A. For convenience of description, the above mutation loci tested by primer combinations are summarized as shown in Table 13.

TABLE 13
Primers and information of the pre-determined loci to be tested

| | Primers corresponding to the tested | | |
|---|---|---|---|
| Gene | locus | locus | HGMD observation |
| GJB2 | GJB2-F1: | 35delG | CD972240 deafness, |
| | CACGCTGCAGACGATCC | | autosomal recessive |
| | GJB2-R1: | | inheritance |
| | CCCCAATCCATCTTCTACTCT | | |
| | GJB2-F2: | 109G > A (VAL37ILE) | CM000016 deafness, |
| | TCCCACATCCGGCTATG | | autosomal recessive |
| | GJB2-R: | | inheritance |
| | GATGGGGAAGTAGTGATCGTAG | 167delT | CD972241 deafness, |
| | | | autosomal recessive |
| | | | inheritance |
| | | 176-191del16 | CD000073 deafness, |
| | | | autosomal recessive |
| | | | inheritance |
| | | 235delC | CD991730 deafness, |
| | | | autosomal recessive |
| | | | inheritance |
| | | 299-300delAT | CD000074 deafness, |
| | | | autosomal recessive |
| | | | inheritance |
| GJB3 | GJB3-F: | 421A > G(ILE141VAL) | CM000019 deafness, |
| | CGTGGACTGCTACATTGCC | | autosomal recessive |
| | GJB3-R: | | inheritance |
| | ATGTTGGGGCAGGGG | 421-423delATT | CD000075 deafness, |
| | | | autosomal recessive |
| | | | inheritance |
| | | 497A-G (ASN166SER) | CM090826 deafness, |
| | | | non-syndrome, |
| | | | autosomal recessive |
| | | | inheritance |
| | | 538C > T | CM980934 deafness, |
| | | (ARG180TER) | non-syndrome, |
| | | | autosomal dominant |
| | | 547G > A | CM980935 deafness, |
| | | (GLU183LYS) | non-syndrome, |
| | | | autosomal dominant |
| | | 580G > A | CM090827 deafness, |
| | | (ALA194THR) | non-syndrome, |
| | | | autosomal recessive |
| | | | inheritance |
| SCL26A4 | PDS2-F: | 281C > T | CM074541 LVAS |
| | CGTCATTTCGGGAGT | | |
| | PDS2-R: | | |
| | CTAAGCAGCCATTC | | |
| | PDS5-F: | 589G > A | CM074557 LVAS |
| | CCCTGACTCTGCTGG | | |
| | PDS5-R: | | |
| | CACTGGCAATCAGGA | | |
| | PDS7-F2: | IVS7-2 A > G | CS991479 deafness |
| | TGGCAGTAGCAATTATCGTC | | syndrome |
| | PDS7-R2: | | Pendred syndrome |
| | TTTCATATGGAGCCAACCTG | | |
| | PDS10-F: | 1174 A > T (N392Y) | CM030959 deafness, |
| | CCACTGCTCTTTCCCGC | | non-syndrome, |
| | PDS10-R: | | autosomal recessive |
| | CAAGAGAAGAATCCTGAGAAGATG | | inheritance |
| | | 1226G > A | CM981503 deafness |
| | | | syndrome |
| | | 1229 C > T(T410M) | CM981504 deafness |
| | | | syndrome |
| | PDS17-F: | 1975G > C | CM073354 deafness, |
| | TTCCTGGACGTTGTTGGAG | | non-syndrome, |
| | PDS17-R: | | autosomal recessive |
| | GATATAGCTCCACAGTCAAGCAC | | inheritance |
| | | 2027 T > A (L676Q) | CM030963 deafness, |
| | | | non-syndrome, |
| | | | autosomal recessive |
| | | | inheritance |
| | PDS19-F: | 2162C > T (T721M) | CM991031 deafness, |
| | TCTTGAGATTTCACTTGGTT | | non-syndrome, |
| | PDS19-R: | | autosomal recessive |
| | GTTCCATTTTAGAAACGGTA | | inheritance |
| | | 2168 A > G | CM981513 deafness |
| | | (HIS723ARG) | syndrome |
| | | IVS15 + 5G > A | CS050413 LVAS |

### Experiment Results:

TABLE 14

| | Information provided by | | | | | | Conclusion of non-invasive | |
|---|---|---|---|---|---|---|---|---|
| | hospital | | Results of non-invasive detection | | | | detection | |
| | Maternal | | | mutated | Total | | Maternal | |
| Sample | genotype | Fetal genotype | mutation | templates¹ | templates² | % | genotype | Fetal genotype |
| Pregnant | No mutation | GJB2 109 G > A | GJB2 109 G > A | 16 | 331 | 4.83% | No mutation | GJB2 109G > A |
| woman 1 | was observed. | heterologous | SLC26A4 | 0 | 376 | 0.00% | was observed. | fetus is |
| | | mutation | IVS7-2A > G | | | | | heterologous. |
| | | | GJB2 | 0 | 162 | 0.00% | | |
| | | | 299-300delAT | | | | | |
| | | | GJB3 538C > T | 0 | 262 | 0.00% | | |
| Pregnant | GJB2 109 G > A | GJB2 109 G > A | GJB2 109 G > A | 78 | 159 | 49.06% | GJB2 109 G > A | GJB2 109G > A |
| woman 2 | heterologous | heterologous | SLC26A4 | 0 | 198 | 0.00% | Mother is | fetus is |
| | mutation | mutation | IVS7-2A > G | | | | heterologous. | heterologous. |
| | | | GJB2 | 0 | 72 | 0.00% | | |
| | | | 299-300delAT | | | | | |
| | | | GJB3 538C > T | 0 | 162 | 0.00% | | |
| Pregnant | GJB2 109 G > A | GJB2 109 G > A | GJB2 109 G > A | 177 | 343 | 51.60% | GJB2 109 G > A | GJB2 109 G > A |
| woman 3 | heterologous | heterologous | SLC26A4 | 0 | 425 | 0.00% | Mother is | fetus is |
| | mutation | mutation | IVS7-2A > G | | | | heterologous. | heterologous. |
| | | | GJB2 | 0 | 132 | 0.00% | | |
| | | | 299-300delAT | | | | | |
| | | | GJB3 538C > T | 0 | 362 | 0.00% | | |
| Pregnant | GJB2 109 G > A | GJB2 109 G > A | GJB2 109 G > A | 52 | 93 | 55.91% | GJB2 109 G > A | GJB2 109 G > A |
| woman 4 | heterologous | Homozygous | SLC26A4 | 0 | 137 | 0.00% | Mother is | Fetus is |
| | mutation | mutation | IVS7-2A > G | | | | heterologous. | homozygous. |
| | | | GJB2 | 0 | 41 | 0.00% | | |
| | | | 299-300delAT | | | | | |
| | | | GJB3 538C > T | 1 | 97 | 1.03% | | |
| Pregnant | SLC26A4 | SLC26A4 | GJB2 109 G > A | 0 | 67 | 0.00% | SLC26A4 | SLC26A4 |
| woman 5 | IVS7-2I | IVS7-2 | SLC26A4 | 59 | 114 | 51.75% | IVS7-2A > G | IVS7-2A > G |
| | heterologous | heterologous | IVS7-2A > G | | | | Mother is | fetus is |
| | mutation | mutation | GJB2 | 0 | 28 | 0.00% | heterologous. | heterologous. |
| | | | 299-300delAT | | | | | |
| | | | GJB3 538C > T | 0 | 71 | 0.00% | | |
| Pregnant | GJB2 109 G > A | No mutation | GJB2 109 G > A | 383 | 837 | 45.76% | GJB2 109 G > A | No mutation |
| woman 6 | heterologous | was observed. | SLC26A4 | 0 | 958 | 0.00% | Mother is | was observed. |
| | mutation | | IVS7-2A > G | | | | heterologous. | |
| | | | GJB2 | 0 | 381 | 0.00% | | |
| | | | 299-300delAT | | | | | |
| | | | GJB3 538C > T | 0 | 702 | 0.00% | | |
| Pregnant | No mutation | GJB2 109 G > A | GJB2 109 G > A | 10 | 122 | 8.20% | No mutation | GJB2 109 G > A |
| woman 7 | was observed. | heterologous | SLC26A4 | 0 | 157 | 0.00% | was observed. | fetus is |
| | | mutation | IVS7-2A > G | | | | | heterologous. |
| | | | GJB2 | 0 | 43 | 0.00% | | |
| | | | 299-300delAT | | | | | |
| | | | GJB3 538C > T | 1 | 107 | 0.93% | | |
| Pregnant | No mutation | GJB2 | GJB2 109 G > A | 0 | 291 | 0.00% | No mutation | GJB2 |
| woman 8 | was observed. | 299-300delAT | SLC26A4 | 0 | 381 | 0.00% | was observed. | 299-300delAT |
| | | heterologous | IVS7-2A > G | | | | | fetus is |
| | | mutation | GJB2 | 14 | 149 | 9.40% | | heterologous. |
| | | | 299-300delAT | | | | | |
| | | | GJB3 538C > T | 0 | 255 | 0.00% | | |
| Pregnant | No mutation | GJB2 109 G > A | GJB2 109 G > A | 34 | 627 | 5.42% | No mutation | GJB2 109 G > A |
| woman 9 | was observed. | heterologous | SLC26A4 | 0 | 748 | 0.00% | was observed. | fetus is |
| | | mutation | IVS7-2A > G | | | | | heterologous. |
| | | | GJB2 | 0 | 271 | 0.00% | | |
| | | | 299-300delAT | | | | | |
| | | | GJB3 538C > T | 0 | 506 | 0.00% | | |
| Pregnant | GJB2 109 G > A | No mutation | GJB2 109 G > A | 79 | 169 | 46.75% | GJB2 109 G > A | No mutation |
| woman 10 | heterologous | was observed. | SLC26A4 | 0 | 250 | 0.00% | Mother is | was observed. |
| | mutation | | IVS7-2A > G | | | | heterologous. | |
| | | | GJB2 | 0 | 75 | 0.00% | | |
| | | | 299-300delAT | | | | | |
| | | | GJB3 538C > T | 0 | 188 | 0.00% | | |
| Pregnant | GJB2 109 G > A | GJB2 109 G > A | GJB2 109 G > A | 117 | 233 | 50.21% | GJB2 109 G > A | GJB2 109 G > A |
| woman 11 | heterologous | heterologous | SLC26A4 | 0 | 265 | 0.00% | Mother is | fetus is |
| | mutation | mutation | IVS7-2A > G | | | | heterologous. | heterologous. |
| | | | GJB2 | 0 | 83 | 0.00% | | |
| | | | 299-300delAT | | | | | |
| | | | GJB3 538C > T | 0 | 205 | 0.00% | | |
| Pregnant | GJB3 538C > T | No mutation | GJB2 109 G > A | 0 | 287 | 0.00% | GJB3 538C > T | No mutation |
| woman 12 | heterologous | was observed. | SLC26A4 | 0 | 354 | 0.00% | Mother is | was observed. |
| | mutation | | IVS7-2A > G | | | | heterologous. | |
| | | | GJB2 | 0 | 130 | 0.00% | | |
| | | | 299-300delAT | | | | | |
| | | | GJB3 538C > T | 124 | 292 | 42.47% | | |
| Pregnant | No mutation | No mutation | GJB2 109 G > A | 0 | 465 | 0.00% | No mutation | No mutation |
| woman 13 | was observed. | was observed. | SLC26A4 | 0 | 577 | 0.00% | was observed. | was observed. |
| | | | IVS7-2A > G | | | | | |
| | | | GJB2 | 0 | 231 | 0.00% | | |
| | | | 299-300delAT | | | | | |
| | | | GJB3 538C > T | 0 | 447 | 0.00% | | |
| Pregnant | No mutation | No mutation | GJB2 109 G > A | 0 | 388 | 0.00% | No mutation | No mutation |
| woman 14 | was observed. | was observed. | SLC26A4 | 0 | 470 | 0.00% | was observed. | was observed. |
| | | | IVS7-2A > G | | | | | |
| | | | GJB2 | 0 | 176 | 0.00% | | |
| | | | 299-300delAT | | | | | |
| | | | GJB3 538C > T | 2 | 313 | 0.64% | | |
| Pregnant | No mutation | No mutation | GJB2 109 G > A | 0 | 248 | 0.00% | No mutation | No mutation |
| woman 15 | was observed. | was observed. | SLC26A4 | 0 | 283 | 0.00% | was observed. | was observed. |
| | | | IVS7-2A > G | | | | | |
| | | | GJB2 | 0 | 82 | 0.00% | | |
| | | | 299-300delAT | | | | | |
| | | | GJB3 538C > T | 1 | 219 | 0.46% | | |
| Pregnant | No mutation | No mutation | GJB2 109 G > A | 0 | 84 | 0.00% | No mutation | No mutation |
| woman 16 | was observed. | was observed. | SLC26A4 | 0 | 94 | 0.00% | was observed. | was observed. |
| | | | IVS7-2A > G | | | | | |
| | | | GJB2 | 0 | 35 | 0.00% | | |
| | | | 299-300delAT | | | | | |
| | | | GJB3 538C > T | 0 | 64 | 0.00% | | |
| Pregnant | No mutation | No mutation | GJB2 109 G > A | 0 | 275 | 0.00% | No mutation | No mutation |
| woman 17 | was observed. | was observed. | SLC26A4 | 0 | 380 | 0.00% | was observed. | was observed. |
| | | | IVS7-2A > G | | | | | |
| | | | GJB2 | 0 | 86 | 0.00% | | |
| | | | 299-300delAT | | | | | |
| | | | GJB3 538C > T | 0 | 304 | 0.00% | | |
| Pregnant | No mutation | No mutation | GJB2 109 G > A | 0 | 364 | 0.00% | No mutation | No mutation |
| woman 18 | was observed. | was observed. | SLC26A4 | 0 | 481 | 0.00% | was observed. | was observed. |
| | | | IVS7-2A > G | | | | | |
| | | | GJB2 | 0 | 163 | 0.00% | | |
| | | | 299-300delAT | | | | | |
| | | | GJB3 538C > T | 0 | 385 | 0.00% | | |

Notes:
¹Mutated templates: the number of templates of the corresponding mutated loci in the non-invasive prenatal detection.
²Total templates: the total number of templates of the corresponding loci in the non-invasive prenatal detection.

Main reagents include: QIAamp Circulating Nucleic Acid Kit, T4 DNA phosphorylation buffer (10×), 10 μM dNTP mixture, T4 DNA polymerase, T4 DNA phosphorylase, dATP solution, Quick ligation buffer (5×), Y-type DNA double strand linkers, T4 DNA Ligase(HC), Quick T4 DNA ligase (NEB), Phusion DNA polymerase (Phusion DNA polymerase mixture), pre-amplification primers, Ultra-pure water, 10× Taq ligase Buffer, Taq ligase, 10× NEBuffer 1, Exo III.

The data standard for the conclusion of the experiment results:

According to an internal statistics, the average amount of fetal plasma DNA in a pregnant woman at gestational age of 12-26 weeks is around 10% based on the amount of plasma DNA of the pregnant woman. Deafness gene disorder is autosomal inherited, and thus the data standard of various combinations of maternal and fetal pathogenic genotypes can be obtained based on the laws of inheritance. The experiment conclusion is deduced based on the genotype data of the tested pathogenic genotypes.

| Genotypes | | Theoretical values | | |
|---|---|---|---|---|
| Mother | Fetus | mother | fetus | total |
| Homozygous mutation | Heterologous mutation | 90% | 10% | 100% |
| Homozygous mutation | Heterologous mutation | 90% | 5% | 95% |
| Heterologous mutation | Homozygous mutation | 45% | 10% | 55% |
| Heterologous mutation | Heterologous mutation | 45% | 5% | 50% |
| Heterologous mutation | Wild type | 45% | 0% | 45% |
| Wild type | Heterologous mutation | 0% | 5% | 5% |
| Wild type | Wild type | 0% | 0% | 0% |

1. A method for non-invasively detecting fetal deafness pathogenic gene mutations, comprising:
(a) designing primers according to the mutations of pre-determined loci of deafness pathogenic genes;
(b) extracting plasma DNAs from a pregnant woman;
(c) connecting the extracted plasma DNAs with pre-amplification linkers to obtain connected products;
(d) PCR pre-amplifying the connected products to obtain pre-amplified products;
(e) cyclizing the pre-amplified products to obtain cyclised DNAs;
(f) PCR amplifying the cyclised DNAs using the designed primers to obtain amplified products; and
(g) high throughput sequencing the amplified products and analyzing the mutations of the fetal deafness pathogenic genes.

2. The method according to claim 1, characterized in that the mutations of pre-determined loci are at least one gene mutation selected from 22 loci of GJB2 gene, GJB3 gene, and SLC26A4 gene.

3. The method according to claim 1, characterized in that the primers are a pair of primers that are backward extended.

4. The method according to claim 1, characterized in that the backward extended pair of primers are designed to aim at exon 2 of GJB2, exon 2 of GJB3, or exon 3, exon 5, exon 7, intron 7, exon 8, exon 10, exon 17 or exon 19 of SLC26A4.

5. The method according to claim 1, characterized in that the backward extended pair of primers are consist of backward extended pair of primers that are adjacent to a group of disease detection loci (e.g. high frequency mutation loci).

6. The method according to claim 1, characterized in that the backward extended pair of primers contain universal primer region suitable for different high-throughput sequencing platforms.

7. The method according to any one of claims 3-6, characterized in that the sequences of the backward extended pair of primers are as follows respectively:

| | |
|---|---|
| GJB2-F1 (SEQ ID NO: 2): | CACGCTGCAGACGATCC |
| GJB2-R1 (SEQ ID NO: 3): | CCCCAATCCATCTTCTACTCT |
| GJB2-F2 (SEQ ID NO: 4): | TCCCACATCCGGCTATG |
| GJB2-R2 (SEQ ID NO: 5): | GATGGGGAAGTAGTGATCGTAG |
| GJB3-F (SEQ ID NO: 7): | CGTGGACTGCTACATTGCC |
| GJB3-R (SEQ ID NO: 8): | ATGTTGGGGCAGGGG |
| PDS3-F (SEQ ID NO: 10): | CGTCATTTCGGGAGT |
| PDS3-R (SEQ ID NO: 11): | CTAAGCAGCCATTCC |
| PDS5-F (SEQ ID NO: 13): | CCCTGACTCTGCTGG |
| PDS5-R (SEQ ID NO: 14): | CACTGGCAATCAGGA |
| PDS7-F2 (SEQ ID NO: 16): | TGGCAGTAGCAATTATCGTC |
| PDS7-R2 (SEQ ID NO: 17): | TTTCATATGGAGCCAACCTG |
| PDS10-F (SEQ ID NO: 19): | CCACTGCTCTTTCCCGC |
| PDS10-R (SEQ ID NO: 20): | CAAGAGAAGAATCCTGAGAAGATG |
| PDS17-F (SEQ ID NO: 22): | TTCCTGGACGTTGTTGGAG |
| PDS17-R (SEQ ID NO: 23): | GATATAGCTCCACAGTCAAGCAC |
| PDS19-F (SEQ ID NO: 25): | TCTTGAGATTTCACTTGGTT |
| PDS19-R (SEQ ID NO: 26): | GTTCCATTTTAGAAACGGTA. |

8. The method according to any one of claims 3-6, characterized in that one pair of the backward extended pair of primers detects one or more loci.

9. The method according to claim 2, characterized in that detection of the 22 loci of GJB2 gene, GJB3 gene and SLC26A4 gene is accomplished in one PCR using 9 pairs of primers.

10. The method according to claim 1, characterized in that the linkers are barcode (multiple sequence labelled) linkers.

11. The method according to claim 10, characterized in that there are at least two base differences between the linkers.

12. The method according to claim 1, characterized in that the linkers are partially matched Y-type linkers.

13. The method according to claim 1, characterized in that the method used in the cyclization of step c) is a splint cyclization, wherein single stand DNAs complementary to both ends of the pre-amplified DNA are used as splints; and close of the ring is completed by a heat-resistant Taq ligase.

14. The method according to claim 1, characterized in that the cyclization of step c) is multiple reactions of a single system circulation consisting of DNA denaturation, splint DNA annealing and connecting.

15. The method according to claim 1, further comprises digesting the uncyclized linear DNA.

16. The method according to claim 1, characterized in that the deafness pathogenic genes have insertion, deletion, substitution or gene fusion mutations.

17. A kit for non-invasively detecting fetal deafness pathogenic gene mutations, comprising: reagents for extracting plasma DNAs, a DNA cyclase, primers and reagents for amplifying target DNAs.

18. The kit according to claim 17, characterized in that the kit further comprises primers and reagents for pre-amplifying the pre-determined loci of deafness pathogenic genes.

19. The kit according to claim 17, characterized in that the kit further comprises reagents for high throughput sequencing.

20. The kit according to claim 17, characterized in that the mutations of pre-determined loci are at least one gene mutation selected from 22 loci of GJB2 gene, GJB3 gene, and SLC26A4 gene.

21. The kit according to claim 17, characterized in that the primers for pre-amplifying the pre-determined loci of deafness pathogenic genes are a pair of primers that are backward extended.

22. The kit according to claim 17, characterized in that the backward extended pair of primers are designed to aim at exon 2 of GJB2, exon 2 of GJB3, or exon 3, exon 5, exon 7, intron 7, exon 8, exon 10, exon 17 or exon 19 of SLC26A4.

23. The kit according to claim 17, characterized in that the backward extended pair of primers are consist of backward extended pair of primers that are adjacent to a group of disease detection loci (e.g. high frequency mutation loci).

24. The kit according to claim 17, characterized in that the backward extended pair of primers contain universal primer region suitable for different high-throughput sequencing platforms.

25. The kit according to any one of claims 21-24, characterized in that the sequences of the backward extended pair of primers are as follows respectively:

| | |
|---|---|
| GJB2-F1 (SEQ ID NO: 2): | CACGCTGCAGACGATCC |
| GJB2-R1 (SEQ ID NO: 3): | CCCCAATCCATCTTCTACTCT |
| GJB2-F2 (SEQ ID NO: 4): | TCCCACATCCGGCTATG |
| GJB2-R2 (SEQ ID NO: 5): | GATGGGGAAGTAGTGATCGTAG |
| GJB3-F (SEQ ID NO: 7): | CGTGGACTGCTACATTGCC |
| GJB3-R (SEQ ID NO: 8): | ATGTTGGGGCAGGGG |
| PDS3-F (SEQ ID NO: 10): | CGTCATTTCGGGAGT |
| PDS3-R (SEQ ID NO: 11): | CTAAGCAGCCATTCC |
| PDS5-F (SEQ ID NO: 13): | CCCTGACTCTGCTGG |
| PDS5-R (SEQ ID NO: 14): | CACTGGCAATCAGGA |
| PDS7-F2 (SEQ ID NO: 16): | TGGCAGTAGCAATTATCGTC |
| PDS7-R2 (SEQ ID NO: 17): | TTTCATATGGAGCCAACCTG |
| PDS10-F (SEQ ID NO: 19): | CCACTGCTCTTTCCCGC |
| PDS10-R (SEQ ID NO: 20): | CAAGAGAAGAATCCTGAGAAGATG |
| PDS17-F (SEQ ID NO: 22): | TTCCTGGACGTTGTTGGAG |
| PDS17-R (SEQ ID NO: 23): | GATATAGCTCCACAGTCAAGCAC |
| PDS19-F (SEQ ID NO: 25): | TCTTGAGATTTCACTTGGTT |
| PDS19-R (SEQ ID NO: 26): | GTTCCATTTTAGAAACGGTA. |

26. The kit according to any one of claims 21-24, characterized in that one pair of the backward extended pair of primers detects one or more loci.

27. The kit according to claim 20, characterized in that detection of the 22 loci of GJB2 gene, GJB3 gene and SLC26A4 gene is accomplished in one PCR using 9 pairs of primers.

28. The kit according to claim 17, characterized in that the plasma DNAs are connected with linkers.

29. The kit according to claim 28, characterized in that the linkers are barcode (multiple sequence labelled) linkers.

30. The kit according to claim 28, characterized in that there are at least two base differences between the linkers.

31. The kit according to claim 28, characterized in that the linkers are partially matched Y-type linkers.

32. The kit according to claim 17, characterized in that the deafness pathogenic genes have insertion, deletion, substitution or gene fusion mutations.
